(19) United States
(12) Patent Application Publication
(10) Pub. No.: US 2008/0233067 A1
(43) Pub. Date: Sep. 25, 2008

(54) Method Of Making Enhanced Efficacy Antiperspirant Actives

(75) Inventors: Wilson Lee, Bloomfield, NJ (US); Xiaozhong Tang, Bridgewater, NJ (US); John Brahms, Piscataway, NJ (US); James Cush, Washington Twp., NJ (US); Anthony Esposito, Roselle, NJ (US); Marie Johansson, Watchung, NJ (US)

Correspondence Address: COLGATE-PALMOLIVE COMPANY, 909 RIVER ROAD, PISCATAWAY, NJ, 08855, US

(73) Assignee: COLGATE-PALMOLIVE COMPANY, NEW YORK, NY (US)

(21) Appl. No.: 12/106,700

(22) Filed: Apr. 21, 2008

Related U.S. Application Data
(63) Continuation of application No. 10/228,328, filed on Aug. 26, 2002.
Application No. 10/228,328 is a continuation-in-part of application No. 09/597,322, filed on Jun. 19, 2000, now abandoned.

(51) Int. Cl. A61K 8/26; A61K 8/28; B02C 23/18

(52) U.S. Cl. 424/66; 424/68; 241/15

(57) ABSTRACT

This invention comprises: (1) a wet grinding method for enhancing the activity of an aluminum or an aluminum/zirconium salt without the dilution and heating traditionally required wherein the enhancement is described as forming a salt wherein the amount of smaller aluminum species as represented by Peak 4+Peak 5 is increased by an amount of at least 10% over the parent salt; and, if zirconium is present, the area of Peak 1 in the parent salt is at least 10% greater than the area of Peak 1 after grinding; (2) an enhanced aluminum or aluminum/zirconium salt itself, and (3) anhydrous (less than 4% water excluding waters of hydration for the enhanced salt) antiperspirant and/or deodorant products made with the salts described in (2).

### CROSS REFERENCE TO RELATED APPLICATIONS

This application is a continuation of U.S. Ser. No. 10/228,328, filed 26 Aug. 2002, which is a continuation in part of U.S. Ser. No. 09/597,322, filed 19 Jun. 2000, both of which are incorporated herein by reference.

### FIELD OF THE INVENTION

This invention relates to the formation of enhanced antiperspirant salts containing (1) aluminum or (2) aluminum and zirconium polymeric species, the salts themselves and cosmetic compositions formulated with such salts. In particular, a wet grinding method has been developed which creates improved antiperspirant salts as reflected in molecular weight distributions for Peaks 1-5 in an SEC chromatogram evidencing a quantitative increase in the smaller species for both aluminum and zirconium species.

### BACKGROUND OF THE INVENTION

Antiperspirant salts, such as aluminum chlorohydrex (also called aluminum chlorohydrex polymeric salts and abbreviated here as “ACH”) and aluminum zirconium glycine salts (abbreviated here as “ZAG”, “ZAG complexes” or “AZG”), are known to contain a variety of polymeric and oligomeric species with molecular weights (MW) ranging from 100-500,000. It has been clinically shown that, in general, the smaller the species, the higher the efficacy for reducing sweat.

In an attempt to increase the quality and quantity of smaller aluminum and/or zirconium species, a number of efforts have focused on (1) how to select the components of ACH and ZAG which affect the performance of these materials as antiperspirants and deodorants; and (2) how to manipulate these components to obtain and/or maintain the presence of smaller types of these components. These attempts have included the development of analytical techniques. Size exclusion chromatography (“SEC”) or gel permeation chromatography (“GPC”) are methods frequently used for obtaining information on polymer distribution in antiperspirant salt solutions. With appropriate chromatographic columns, at least five distinctive groups of polymer species can be detected in a ZAG, appearing in a chromatogram as peaks 1, 2, 3, 4 and a peak known as “5”. Peak 1 is the larger Zr species (greater than the pore size of column materials, (particularly greater than 120-125 Angstroms). Peak 2 is the larger aluminum species (particularly greater than 120-125 Angstroms). Peak 3 is the medium species. Peak 4 is the smaller aluminum species (aluminum oligomers), and has been particularly correlated with enhanced efficacy for both ACH and ZAG salts.

Peak 5 (sometimes referred to as Peak 5-6) is the smallest aluminum species. The retention time (“Kd”) for each of these peaks varies depending on the experimental conditions. Various analytical approaches for characterizing the peaks of ACH and various types of ZAG actives are found in “Antiperspirant Actives—Enhanced Efficacy Aluminum-Zirconium-Glycine (AZG) Salts” by Dr. Allan H. Rosenberg (Cosmetics and Toiletries Worldwide, Fondots, D. C. ed., Hartfordshire, UK: Aston Publishing Group, 1993, pages 252, 254-256). Using GPC, Rosenberg describes four peaks identified as Al Kd 0.0; 0.24; 0.40; and 0.60. Activated ACH is identified as material having an enriched Al Kd 0.4 content. Spray drying AZG within a prescribed time frame to fix the desired distributions of the 4 peaks in a powder has also been suggested in the same reference Rosenberg, A., “New Antiperspirant Salt Technology” (Cosmetics and Toiletries Worldwide, Fondots, D. C. ed., Hartfordshire, UK: Aston Publishing Group, 1993, pages 214-218).

Other techniques have been developed as well such as size exclusion chromatography (“SEC”) sometimes referred to as gel permeation chromatography (“GPC”) (depending on the type of column used) which can utilize SEC columns in HPLC systems. A combination system combining inductively coupled plasma (“ICP”) with SEC for an SEC-ICP system has also been developed. Such techniques can be used to investigate whether zirconium and aluminum species co-elute at similar retention times or elute separately from the column at different retention times. In a particular method the SEC and ICP equipment are linked to characterize and monitor the zirconium and aluminum content and species in an aqueous solution of zirconium and aluminum, especially ZAG solutions. This is useful to investigate whether zirconium and aluminum species co-elute at similar retention times or elute separately from the column at different retention times.

Attempts to activate antiperspirant salts with improved efficacy have included developing processes for obtaining better types of ACH such as by heating solutions of ACH with or without elevated pressure in order to depolymerize larger aluminum species into Peak 4 species. Examples can be found in U.S. Pat. No. 4,359,456 to Gosling et al. Since ACH solutions may be used as starting materials for aluminum zirconium glycine (ZAG or AZG) salts, heating ACH solutions has also been used to enrich Peak 4 oligomers before spray drying.

U.S. Pat. No. 4,775,528 to Callaghan et al describes the formation of a solid antiperspirant composition having an Al:Zr atomic ratio from 6:1 to 1:1; the GPC profile of the antiperspirant in solution gave a ratio of at least 2:1 for peak 4/peak 3. This reference specifies that the zirconyl hydrochloride be mixed with the aluminum chlorhydroxide solution before the drying step is completed. The emphasis is placed on optimizing the aluminum chemistry and there is no discussion of any effects on the zirconium chemistry. Likewise, U.S. Pat. No. 4,871,525 to Giovanniello, et al. also teaches a method to activate ZAG by thermally enriching the Al Kd 0.4 content in aqueous solutions.

Such approaches do not, however, directly address the issue of zirconium species. Rosenberg points out that activated AZG salts with enriched Al Kd 0.4 content do not necessarily give enhanced performance in antiperspirant use and notes that zirconium polymer distributions are more important than Al Kd 0.4 enrichment in predicting clinical efficacy, with lower molecular weight zirconium polymer distributions being more desirable.

The dilution/heating process which is normally used to activate the aluminum species involves heating a dilute aqueous solution of the antiperspirant salt and then spray drying the material to a powder form. This technique depolymerizes the aluminum. Unfortunately the technique that is used to increase the amount of small to medium aluminum species works in a counterproductive way to reduce the efficacy of the zirconium species by polymerizing the zirconium. Unlike aluminum, which can be depolymerized by the heating and dilution before spray-drying described above, the polymerization of the zirconium species is irreversible. Heretofore, the best that could be done was to minimize the polymerization of the zirconium species during processing.

Attempts to reduce the problems in the polymerization of zirconium have included the use of glycine in antiperspirant salts to control the polymerization of zirconium species. For example, European patent Application 0 499 456 A2 assigned to Bristol-Myers Squibb Company describes a ZAG complex and a process for making the complex comprising mixing zirconium hydroxychloride, a selected aluminum chloro species and an amino acid in aqueous solution and, optionally drying the aqueous solution to obtain a dry ZAG salt.

European Patent Application EP 0 653 203 A1 to Rosenberg et al describes a process for making ZAG salt with high antiperspirant activity. According to this reference, glycine is added to Zr starting materials at ambient temperature, and the mixed Zr/glycine is amixed with the aluminum chlorohydrate starting material immediately prior to spray drying in a continuous or semi-continuous operation.

U.S. Pat. No. 4,871,525 to Giovanniello et al describes a solid powder of aluminum zirconium hydroxyl halide glycinate complex having improved antiperspirant activity wherein the glycine is used to prevent gel formation. The ratio of Zr to glycine is less than 1:1.

In general, it has been found that large or medium size aluminum polymeric species (Peak 2 and Peak 3 species) in antiperspirant salts can be converted to smaller ones (Peak 4) by diluting an aqueous solution of the salt to a concentration of about 2-20% (w/w), and heating the diluted solution to a temperature of about 90° C. for a period of time. (Peak 5 or Peak 5-6 have not usually been mentioned because chemical equilibrium factors in aqueous solutions have limited the ability to increase this peak.) However, there has been no thermal activation method available to convert large zirconium species into small ones. It has only been possible to prevent small zirconium species from polymerizing by forming complexes with amino acids or with salts thereof.

With regard to making smaller particle sized antiperspirant salts, reference is made to U.S. Pat. No. 5,098,698 to Kawam et al and U.S. Pat. No. 4,987,243 to Kaw am et al both describe a process for preparing submicron antiperspirant adduct wherein the first step is dissolving a mixture of an aluminum-containing salt and a stearic stabilizer in a solvent. U.S. Pat. No. 5,864,923 to Rouanet et al and U.S. Pat. No. 5,725,836 teach the use of supercritical fluids to form aerogels.

Even if modification of current spray drying processes is used, spray drying a solution of antiperspirant salt immediately to remove water would result in an anhydrous powder with the same polymer distribution of aluminum and zirconium species in the solution. The finest powder commercially available has a particle size distribution from 2-10 microns with average size of about 7 microns as made by a dry-grinding method.

It has now been found that an antiperspirant salt containing aluminum or aluminum and zirconium can be activated by converting both large aluminum and zirconium polymers into small ones without the use of heating or dilution or the need for the special last minute addition of the zirconium component. One of the most significant features of this invention is that it is the first time that a process for activating a zirconium salt has been discovered.

### SUMMARY OF THE INVENTION

This invention comprises:

(1) a method for enhancing the activity of an aluminum or an aluminum/zirconium salt without the dilution and heating traditionally required wherein the enhancement is described as forming a salt wherein amount of smaller aluminum species as represented by Peak 4+Peak 5 is increased by an amount of at least 10% (particularly by an amount of at least 20% and, even more particularly, by an amount of at least 25%) over the parent salt; and, if zirconium is present, the area of Peak 1 in the parent salt, i.e. before grinding, is at least 10% greater (particularly 20% greater and, more particularly, 25% greater) than the area of Peak 1 after grinding;
(2) an enhanced aluminum or aluminum/zirconium salt itself; and
(3) anhydrous (less than 4% water excluding waters of hydration for the enhanced salts) antiperspirant and/or deodorant products made with the salts described in (2).

Using this method, an antiperspirant salt containing aluminum and, optionally, zirconium, is mixed with a non-aqueous (for example, a non-aqueous and hydrophobic) liquid vehicle in which the salt is suspended but not appreciably soluble (less than 1.0%) and then ground at a temperature in the range of 20-70 degrees C. to an average particle size of less than or equal to 2 microns, particularly less than or equal to 1.5 microns. The process is carried out without the use of added water or external heating.

The invention also includes salts made by the described process and formulations of anhydrous antiperspirants and/or deodorants made with the salts in stick, gel, cream, soft solid, roll-on and aerosol products.

### DESCRIPTION OF THE DRAWINGS

FIG. 1 shows SEC profiles for 10% solutions of a salt, REACH AZP-908 aluminum zirconium tetrachlorohydrex gly (Reheis Inc., Berkeley Heights, N.J.). Chromatogram (a), represented by the dashed line, shows a SEC profile of the salt before grinding (mean particle size of 5.882 microns). Chromatogram (b), represented by the dotted line, shows the same salt after grinding as described in Example 2S (mean particle size 1.452 microns). Chromatogram (c), represented by the solid line, shows the salt of (b) after further grinding as described in Example 1P (mean particle size 1.114 microns). These SEC profiles were prepared using the analytical method of Example 1S. The x axis is in minutes and the y axis is in absorption units (relative scale). Peaks 1, 3, 4 and 5 are noted in FIG. 1.

FIG. 2 shows SEC profiles for 10% solutions of a salt, Reach AZZ-902 aluminum zirconium trichlorohydrex gly (Reheis Inc.). Chromatogram (a), represented by the dashed line, shows a SEC profile of the salt before grinding (mean particle size of 5.647 microns). Chromatogram (b), represented by the solid line, shows the same salt after grinding as described in Example 3S (mean particle size 1.036 microns). These SEC profiles were prepared using the analytical method of Example 1S. The x axis is in minutes and the y axis is in absorption units (relative scale). Peaks 1, 3, 4 and 5 are noted in FIG. 2.

FIG. 3 shows SEC profiles for 10% solutions of a salt, REZAL-36 GP aluminum zirconium tetrachlorohydrex gly (Reheis Inc.). Chromatogram (a), represented by the dashed line, shows a SEC profile of the salt before grinding (mean particle size of 6.731 microns). Chromatogram (b), represented by the solid line, shows the same salt after grinding as described in Example 4S (mean particle size 1.651 microns). These SEC profiles were prepared using the analytical method of Example 1S. The x axis is in minutes and the y axis is in absorption units (relative scale). Peaks 1, 3, 4 and 5 are noted in FIG. 3.

### DETAILED DESCRIPTION OF THE INVENTION

Process—The process of the invention may be viewed as affecting both the physical size of the particles of the active salt in powder form and the molecular weight distribution of the various aluminum and zirconium species in the active salt. An antiperspirant salt comprising (a) aluminum or (b) aluminum and zirconium is mixed with a non-aqueous liquid vehicle (for example, a non-aqueous and hydrophobic vehicle) in which the salt is suspended but not appreciably soluble (less than 1.0%) and then ground at a temperature in the range of 20-70 degrees C. to an average particle size of less than or equal to 2 microns, particularly less than or equal to 1.5 microns. The process is carried out without the use of added water or external heating. It should be noted that, in general, the poorer performing parent salts will experience larger increases in smaller aluminum species and larger decreases in larger zirconium species.

The types of aluminum and zirconium based salts that may be processed in this invention include all those which are commonly considered antiperspirant active materials and covered by FDA Monograph as Category I antiperspirant actives and which contain aluminum or aluminum and zirconium. Examples of suitable salts which can be used as starting materials include conventional aluminum and aluminum/zirconium salts, as well as aluminum/zirconium salts complexed with a neutral amino acid such as glycine, as known in the art. See each of European Patent Application Number. 512,770 A1 and PCT case WO 92/19221, the contents of each of which are incorporated herein by reference in their entirety, for disclosure of antiperspirant active materials.

Suitable materials include (but are not limited to) aluminum chlorides (various types including, for example, anhydrous form, hydrated form, etc.), zirconyl hydroxychlorides, zirconyl oxychlorides, basic aluminum chlorides, basic aluminum chlorides combined with zirconyl oxychlorides and hydroxychlorides, and organic complexes of each of basic aluminum chlorides with or without zirconyl oxychlorides and hydroxychlorides and mixtures of any of the foregoing. These include, by way of example (and not of a limiting nature), aluminum chlorohydrate, aluminum chloride, aluminum sesquichlorohydrate, aluminum chlorohydrol-propylene glycol complex, zirconyl hydroxychloride, aluminum-zirconium glycine complex (for example, aluminum zirconium trichlorohydrex gly, aluminum zirconium pentachlorohydrex gly, aluminum zirconium tetrachlorohydrex gly and aluminum zirconium octochlorohydrex gly), aluminum dichlorohydrate, aluminum chlorohydrex PG, aluminum chlorohydrex PEG, aluminum dichlorohydrex PG, aluminum dichlorohydrex PEG, aluminum zirconium trichlorohydrex gly propylene glycol complex, aluminum zirconium trichlorohydrex gly dipropylene glycol complex, aluminum zirconium tetrachlorohydrex gly propylene glycol complex, aluminum zirconium tetrachlorohydrex gly dipropylene glycol complex, and mixtures of any of the foregoing. The aluminum-containing materials can be commonly referred to as antiperspirant active aluminum salts. Generally, the foregoing metal antiperspirant active materials are antiperspirant active metal salts.

A particular group of such antiperspirant actives materials includes aluminum chlorohydrate, aluminum dichlorohyrate, aluminum sesquichlorohydrate, aluminum zirconium trichlorohyrate, aluminum zirconium tetrachlorohyrate, aluminum zirconium pentachlorohyrate, aluminum zirconium octachlorohyrate, aluminum zirconium trichlorohydrex gly, aluminum zirconium tetrachlorohydrex gly, and aluminum zirconium pentachlorohydrex gly.

Another particular group of such antiperspirant actives include, by way of example (and not of a limiting nature), aluminum chlorohydrate, aluminum chloride, aluminum sesquichlorohydrate, zirconyl hydroxychloride, aluminum-zirconium glycine complex (for example, aluminum zirconium trichlorohydrex gly, aluminum zirconium pentachlorohydrex gly, aluminum zirconium tetrachlorohydrex gly and aluminum zirconium octochlorohydrex gly), aluminum chlorohydrex PG, aluminum chlorohydrex PEG, aluminum dichlorohydrex PG, and aluminum dichlorohydrex PEG.

A third particular group of such antiperspirant actives include aluminum zirconium trichlorohydrex and aluminum zirconium tetrachlorohydrex either with or without glycine. A particular antiperspirant active is aluminum trichlorohydrex gly such as Reach AZZ-902 SUF (from Reheis Inc., Berkley Heights, N.J.) which has 98% of the particles less than 10 microns in size, but greater than 3 microns in size.

A fourth particular group of such antiperspirant actives include the enhanced efficacy aluminum salts and the enhanced efficacy aluminum zirconium salt-glycine materials, having enhanced efficacy due to improved molecular distribution, known in the art and discussed, for example, in PCT No. WO92/19221, the contents of which are incorporated by reference in their entirety herein.

More particular examples of such salts include:

### Aluminum Chlorohydrate

Chlorhydrol powder, Reach-101. Reach 301, Reach-501, Westchlor 200, Westchlor DMI 200. Summit ACH-325. Summit ACH-321, and Summit ACH-331.

### Aluminum Zirconium Tetrachlorohydrex-Gly

### Reach AZP-701, Reach AZP-902, Reach AZP-908, Reach AZP-255, Reach AZP-855, Rezal-36, Westchlor ZR 35B, Summit AZG-368, Summit AZG-369 Summnit AZG-370, Summit Q5-7155 AAZG, and Summit Q5-7167 AAZG.

### Aluminum Zirconium Trichlorohydrex-Gly

### Reach AZZ-902, Reach AZZ-855, Reach AZZ-908, Rezal-33, Westchlor ZR 30B, Westchlor ZR 58B, Westchlor ZR 60B, Summit Q5-7160 AZAG, and Summit AZG5-7164.

### Aluminum Zirconium Octachlorohydrex-Gly

### Reach AZO-902, Reach AZO-908, and Westchlor ZR82B.

### Aluminum Zirconium Pentachlorohydrex-Gly

Rezal-67 and Westchlor ZR 80B.

Also, corresponding nitrate, bromide and sulfate salts of any of the foregoing may be used.

In addition, to the Category I active antiperspirant ingredients listed in the Food and Drug Administration's Monograph on antiperspirant drugs for over-the-counter human use, there are other ingredients that can be used, such as tin or titanium salts used alone or in combination with aluminum compounds (for example, aluminum-stannous chlorohydrates), aluminum nitratohydrate and its combination with zirconyl hydroxychlorides and nitrates, can be incorporated as an antiperspirant active ingredient in antiperspirant compositions according to the present invention.

The non-aqueous liquid is used as a vehicle in which the salt is not appreciably dissolved but, in fact, is suspended. Such a liquid vehicle can be from various categories such as:

(a) cosmetic esters (for example, ethoxylates, propoxylates, benzoates, adipates), especially fatty esters having 6-22 carbons in straight or branched chains;

(b) glycols and polyols such as propylene glycol and dipropylene glycol;

(c) volatile silicones such as the cyclomethicones,

(d) non-volatile silicones such as polydimethicone having a viscosity of up to 350 centistokes;

(e) hydrocarbons such as mineral oils;

(f) alcohols having more than three carbons;

(g) mixtures of the foregoing.

Particular examples of such vehicles include the following items in TABLE A.

TABLE A

| Supplier | Tradename | Chemical Name |
|---|---|---|
| Alzo | Dermol 25-3B | C12-C15 ethoxy benzoate |
| Alzo | Dermol 489 | Diethylene Glycol |
| | | dioctanoate/diisononoate |
| Alzo | Dermol 816 | octyl palmitate |
| Alzo | Dermol DIA | diisopropyl adipate |
| Alzo | Dermol DPG- | dipropylene glycol |
| | 2B | dibenzoate |
| Alzo | Dermol G-76 | Glycereth-7 benzoate |
| Alzo | Dermol PGB | propylene glycol benzoate |
| Alzo | Polyderm | glycereth-7 polyurethane |
| | PPI-G7 | |
| Amercol | Fluid AP | PPG-14 Butyl Ether |
| BASF | Lutrol OP- | PPG-26 oleate |
| | 2000 | |
| Bernel | Hetester PHA | propylene glycol |
| | | isoceteth-3 acetate |
| Bernel | Hetester | Propylene Glycol Myristyl |
| | PMA | Ether Acetate |
| Dow Corning | DC 245 | cyclomethicone |
| Dow Corning | DC 345 | cyclomethicone |
| Finetex | Finsolv EMG- | Methyl Gluceth-20 |
| | 20 | Benzoate |
| Finetex | Finsolv PG- | Dipropylene glycol |
| | 22 | dibenzoate |
| Finetex | Finsolv PL- | Poloxamer 105 Benzoate |
| | 355 | |
| Finetex | Finsolv PL-62 | Poloxamer 182 |
| | | Dibenzoate |
| Henkel | Cetiol 868 | octyl stearate |
| ISP | Escalol 597 | octocrylene |
| Lipo | Liponate 2- | PEG-4 diheptanoate |
| | DH | |
| Lipo | Liponate | neopentylglycol |
| | NPGC-2 | dicaprylate/dicaprate |
| Lipo | Liponate PC | propylene glycol |
| | | dicaprylate/dicaprate |
| Lipo | Liponate TDS | tridecyl stearate |
| Phoenix | Pelemol G7B | glycereth-7 benzoate |
| PPG | Macol 57 | PPG-10 butane diol |
| PPG | Masil 756 | tetrabutoxy trisiloxane |
| Rhone Poulenc | benzyl | benzyl benzoate |
| | benzoate | |
| Rhone-Poulenc | Benzyl | benzyl salicylate |
| | Salicylate | |
| Scher | DIPSAL | dipropylene glycol |
| | | salicylate |
| Scher | Schercemol | diisopropyl dimer |
| | DID | dilinoleate |
| Scher | Schercemol | diisostearyl dimer |
| | DISD | dilinoleate |
| Trivent | DIDA | diisodecyl adipate |
| Trivent | DOS | Dioctyl sebacate |
| Trivent | OC-G | tricapylin |
| Trivent | PE-48 | pentaerythritol |
| | | tetraoctanoate |
| Union Camp | Unimate DBS | dibutyl sebacate |
| Union Camp | Unimate DCA | dicapryl adipate |
| Union Camp | Unimate | diisopropyl sebacate |
| | DIPS | |
| Union Camp | Unimate EHP | 2-ethylhexyl palmitate |
| Vevy | Dodecalene | |
| ALZO | Dermol | dipropylene glycol |
| | DPGB | benzoate |
| Alzo | Wickenol 159 | dioctyl succinate |
| Amoco | SilkFlo 364 | polydecene |
| BASF | Luvitol EHO | cetearyl octanoate |
| Bernel | Bernel Ester | isocetyl octanoate |
| | 168 | |
| Bernel | Bernel Ester | octyldodecyl myristate |
| | 2014 | |
| Bernel | Bernel Ester | cetyl octanoate |
| | CO | |
| Bernel | Bernel Ester | dicapryl malleate |
| | DOM | |
| Bernel | Bernel Ester | neopentyl dicaprate |
| | NPDC | |
| Bernel | Dermol 185 | isostearyl neopentanoate |
| Bernel | Hetester HSS | Isocetyl stearoyl stearate |
| Bernel | Hetester ISS | Isostearyl stearoyl |
| | | stearate |
| Bernel | Minno 21 | neopentyl glycol |
| | | dioctanoate/diisostearate |
| Bernel | Minno 41 | neopentyl glycol |
| | | diisostearate/dioctanoate |
| Finetex | Finsolv P | PPG-15 Stearyl Ether |
| | | Benzoate |
| ISP | Escalol 507 | octyl dimethyl PABA |
| ISP | Escalol 557 | octylmethoxycinnamate |
| Phoenix | Pelemol 2022 | octyl dodecyl behenate |
| Trivent | OC-16 | cetyl octanoate |
| Trivent | OL-10B | isodecyl oleate |
| Unichema | Prisorine | 2-ethyl hexyl isostearate |
| | 2036 | |
| Unichema | Prisorine | isostearyl isostearate |
| | 2039 | |
| Union Camp | Unimate IPP | isopropyl palmitate |
| Union Camp | X81-765-16 | Isopropyl Stearate |
| Vevy | Myristol 2-8- | Octyl dodecyl myristate |
| | 12 | |
| Alzo | Dermol PEB | phenoxyethyl benzoate |
| Alzo | Dermol 89 | octyl isononanoate |
| Alzo | Dermol B246 | benzyl laurate/myristate/ |
| | | palmitate |
| Alzo | Dermol ICSA | Isohexadecyl salicylate |
| Alzo | Dermol IDSA | Isodecyl salicylate |
| Alzo | Dermol TDSA | Isotridecyl salicylate |
| Bernel | Bernel Ester | octyl pelargonate |
| | OPG | |
| Bernel | Citmol 316 | triisocetyl citrate |
| Bernel | Dermol 105 | isodecyl neopentanoate |
| Bernel | Dermol 126 | laureth-2 benzoate |
| Bernel | Hetester FAO | C12-C15 Alkyl Octanoate |
| Exxon | Isopar M | isoparaffin |
| Exxon | Isopar V | isoparaffin |
| Exxon | Norpar 15 | normal paraffin |
| Fancor | Fancol ID | isododecane |
| Fancor | Fancol IE | isoeicosane |
| Fancor | Fancol IH | isohexadecane |
| Finetex | Finsolv BOD | Octyl Dodecyl Benzoate |
| Finetex | Finsolv EB | 2-ethyl hexyl benzoate |
| Finetex | Finsolv SB | isostearyl benzoate |
| Finetex | Finsolv TN | C12-C15 alkyl benzoate |
| Henkel | Cetiol OE | dicapryl ether |
| Henkel | Cetiol A | hexyl laurate |
| Henkel | Cetiol S | Dioctylcyclohexane |
| ICI | Brij 30 | laureth-4 |
| ISP | Ceraphyl 41 | C12-C145 alkyl lactate |
| ISP | Escalol 587 | octyl salicylate |
| Jarchem | C12/C14 | C12/C14 alcohol mix |
| | alcohol mix | |
| Jarchem | Jarchol I16 | isocetyl alcohol |
| JarChem | Jarcol I18-T | isostearyl alcohol |
| Jarchem | Jarcol I20 | C20 guerbet alcohol |
| Penta | Hexyl | hexyl benzoate |
| | benzoate | |
| Phoenix | Pelemol IN-2 | isononyl isononanoate |
| Phoenix | Pelemol ISL | isostearyl lactate |
| PPG | Mazon EE-1 | benzyl laurate |
| Presperse | Permethyl | aliphatic hydrocarbons |
| | 102A | |
| Trivent | NP-13 | Tridecyl neopentanoate |
| Trivent | OC-13 | tridecyl octanoate |
| Trivent | OS | octyl salycilate |
| Unichema | Prisorine | isopropyl isostearate |
| | 2021 | |
| Unichema | Prisorine | isostearyl alcohol |
| | 3515 | |
| Union Camp | Harkamex | C12/C14 alcohol mix |
| Union Camp | Unimate IPIS | isopropyl isostearate |
| Witco | Klearol | light mineral oil |
| Witco | PD-23 | petroleum distillate |
| Witco | PD-28 | petroleum distillate |
| | | n-heptane |
| | | 1-octanol |
| | | lauryl alcohol |
| | | isopropyl palmitate |
| | | oleyl alcohol |

Particular examples of vehicles include cyclosiloxane (for example, a cyclomethicone such as D5 cyclomethicone), mineral oils, glycols and polyols, and low viscosity fatty esters having 8-18 carbons.

The glycol or polyglycol is selected from the group consisting of ethylene glycol, propylene glycol, 1,2-propanediol, diethylene glycol, triethylene glycol, tetraethylene glycol, dipropylene glycol, tripropylene glycol, methyl propanediol, 1,6-hexanediol, 1,3-butanediol, 1,4-butanediol, PEG4 through PEG-100, PPG-9 through PPG-34, pentylene glycol, neopentyl glycol, trimethylpropanediol, 1,4-cyclohexanedimethanol, 2,2-dimethyl-1,3-propanediol, 2,2,4,4-tetramethyl-1,3-cyclobutanediol, and mixtures thereof. More particular examples of the glycol component include one or more members of the group consisting of propylene glycol, dipropylene glycol, tripropylene glycol, 2-methyl-1,3-propanediol, methyl propylene glycol, low molecular weight (less than 600) polyethylene glycol, low molecular weight (less than 600) polypropylene glycols, and mixtures of any of the foregoing. Tripropylene glycol has lower irritancy. Mixtures of glycols may be used to balance these desirable properties.

It should also be noted that the viscosity of such vehicle must be considered in relationship to the grinding equipment, with heavier equipment being able to handle higher viscosity materials. Viscosity modifying agents (for example, surfactants) can be added as needed as long as the active salt is not soluble in the viscosity modifying agent.

The processing itself is used to reduce the average particle size so that it does not exceed 2 microns, especially not exceeding 1.5 microns and, more particularly having at least 50% of the particles with a size below 1.10 microns. As described below, enhanced salts can be prepared having an average particle size less than or equal to 0.5 microns with some particles approaching 0.2-0.3 microns.

The process of this invention not only reduces the size of the particles, it also changes the distribution of the molecular species of aluminum and zirconium within the particles. This may be ascertained, for example, by the analytical techniques described herein.

It is important to note that up to this time, there has been no grinding process available that could achieve the small particles described herein without sacrificing the performance of the salts through dehydration and dehydroxylation of the aluminum species and zirconium species and the agglomeration of the particles.

In order to implement the process, appropriate equipment must be used. In selecting appropriate equipment, various choices are available and several processing factors should be considered:

Media Balls—Examples of suitable balls include 0.2 mm-0.4 mm yttrium-stabilized Zirconium Oxide (TZP) for both media hardness and grinding performance. These are commercially available (for example, from Tosoh Ceramics, Japan). Smaller balls may be made or purchased from other sources now or in the near future such as those having a 0.075 size. Other materials include soda lime glass, zirconium toughened alumina and steel.
Mill—Examples of suitable mills include a number of those described in Perry's Chemical Engineering Handbook (7^{th} Edition) as limited by the particle sizes required for the invention (see Tables 20-6 and 20-7 at pages 20-23). Suitable types of size reduction equipment include:
(1) Media Mills such as (a) Ball, pebble, rod and compartment mills (batch and continuous); (b) Autogenous tumbling mills; (c) Stirred ball and bead mills (for example, LME 1 unit from Netzsch Inc. (Exton, Pa.) which incorporates an ultra high molecular weight (UHMW) liner, rotor and rotor shaft to minimize product contamination during the grinding operation as opposed to an all stainless steel mill; and (d) Vibratory mills. Such equipment may be obtained from one or more of the following companies: Draiswerke (Mahwah, N.J.); and Netzsch, Inc. (Exton, Pa.).
(2) Medium peripheral-speed mills such as (a) Ring-roll and bowl mills; (b) Roll mills, cereal type; (c) Roll mills, paint and rubber type; (d) Buhrstones.
(3) High-peripheral-speed mills such as (a) Fine grinding hammer mills; (b) Pin mills; (c) Colloid mills; (d) Wood pulp beaters.

Fluid energy superfine mills such as (a) Centrifugal jet; (b) Opposed jet; (c) Jet with anvil; and (d) Fluidized-bed jet.

Media mill grinding is of particular interest. Media mill grinding uses selected media to accomplish size reduction either as a wet or dry process with the exception of the autogenous tumbling mills which use larger lumps of the material to be ground as the grinding media. With tumbling or vibratory mills, the external vessel provides the motion necessary for the media to accomplish the required grinding. The stirred ball and bead mills use a fixed vessel (sometimes with recirculation loops) and a high speed rotor to achieve the grinding performance required. The LME 1 unit described above is capable of generating 1.0 micron particles when used with the method of this invention. Vibratory mills are also capable of 1.0 micron particle sizes in dry form.

Temperature Control—Much of the energy used in grinding applications evolves into heat. By some estimates up to 98% of grinding energy can be lost as heat. It is preferred that chilled water (for example, in the 0-5 degree C. range) around a jacketed vessel be used to maintain temperature control.
Viscosity Build-Up—Experimental work done for this invention used active-in-silicone systems from 15-40% concentration as the starting material. In all cases significant viscosity increases were observed due to the enormous increase in the surface area of the active particles and subsequent particle attractive forces. Viscosity reduction agents such as lecithin and other surfactants can be used to control the buildup for ease in processing. It is to be noted, however, that this increase in viscosity can also be used to reduce the amount of thickeners or gelling agents needed for the final cosmetic products.

The process is carried out by mixing the active salt with a vehicle selected to be one or more members from the group described above. The salt is not appreciably soluble in the vehicle (less than 5%) and is suspended in the vehicle in a concentration of 15-40% by weight, especially 20-30% and, particularly 25%. The suspension is then ground at a temperature in the range of 20-70 degrees C. to an average particle size of less than or equal to 2 microns, particularly less than or equal to 1.5 microns, especially and preferably where at least 50% by weight of the salt has a particle size below 1.10 microns. The process is carried out without the use of added water or external heating and, in fact, may require cooling to maintain temperature to form the enhanced salts of the invention

The enhancement of the salt can be monitored by certain analytical techniques. Examples of several techniques have been described above as well as in the examples below. These include SEC, GPC and various modifications of such techniques. In one method the SEC or GPC columns separate the aluminum and zirconium species by molecular size, using a photodiode array detector connected to the column outlet. The eluent fractions from the SEC or GPC may be evaluated further by analysis of the individual fractions by ICP. In a second method, (which is used in some of the examples below), SEC may be directly coupled to ICP. The eluent fractions passing through the column are directly linked to the ICP unit; the ICP unit in this case is used as a detector. Data points are collected such as, for example, one data point every 6 seconds. It should be noted that the identity of the peaks using the SEC test described in Example 1S below was previously verified in other work wherein the ICP system was used as a detector. This previous work was done in order to obtain a profile for the antiperspirant active salt. An ICP unit is directly coupled to an HPLC unit in which the column has been selected to be an organically coated silica as an SEC system. The ICP unit is used as a detector so that the oligomeric fractions separated by the SEC column are elucidated on-line quantitatively for Al, Zr and other elements. The ICP's detector is, for example, a simultaneous charge induction device (CID) with a wavelength of 175 to 800 nm. The eluent from the SEC column is analyzed and a data point is noted periodically such as about once every six seconds for Al and Zr. The data points collected are plotted against retention time, to form the chromatogram for each element separately. The number for the individual peak areas represents the relative concentration for that specific element. (See discussion in U.S. Pat. No. 5,997,850.) The method described in Example 1S is a more commercially viable method for a manufacturing environment.

It should be noted that normal detection methods do not measure a related increase in another peak as being associated with a smaller zirconium species. It has been shown that the smaller zirconium species are absorbed on the column. See U.S. Pat. No. 5,997,850. This is verified by reforming the larger zirconium species with dilution. The dilution of the enhanced salt in water causes the larger zirconium species to reform and, thus, Peak 1 will increase to reflect the re-formation of the larger species. It is noted that Peak 1 is exclusively larger zirconium species and the remaining peaks are all aluminum species.

Formulated Products—In its third aspect this invention also includes cosmetic products such as antiperspirants and/or deodorants which are made with the enhanced active salts from the inventive process described above. The formulations of this invention may be made by conventional techniques such as those described in Cosmetics and Toiletries Industry (second edition, 1996) (Chapman and Hall, NY, N.Y.). The enhanced salt is used in place of the normally used active salt, however, mixtures of enhanced salt and traditional salt may be used (for example, because of cost considerations). The use of an enhanced salt of the invention results in improved efficacy, a reduction in the amount of thickener that is needed and improved aesthetics. The activated salts of this inventions can be used in a wide variety of formulations, and in any products which call for the inclusion of antiperspirant salts, provided the formulations are:
(a) anhydrous (no more than 4% water);
(b) do not contain methanol, ethanol or isopropanol in an amount greater than 5%; and
(c) the total amount of glycol component (propylene glycol, dipropylene glycol, tripropylene glycol, polypropylene glycol, etc.) does not exceed 50% by weight of the amount of enhanced antiperspirant active salt in the formulation.

The formulated products of this invention include antiperspirants (where a sufficient amount of salt is added to have an antiperspirant effect) and deodorants (where a lower level of an antiperspirant salt can be used). In traditional compositions antiperspirant actives can be incorporated into compositions in amounts in the range of 0.1-25% of the final composition, the amount used will depend on the formulation of the composition. For example, at amounts in the lower end of the broader range (for example, 0.1-10% on an actives basis), a deodorant effect may be observed. At lower levels the antiperspirant active material will not substantially reduce the flow of perspiration, but will reduce malodor, for example, by acting as an antimicrobial material. At amounts of 10-25% (on an actives basis) such as 15-25%, by weight, of the total weight of the composition, an antiperspirant effect may be observed. The antiperspirant active material is desirably included as particulate matter suspended in the composition of the present invention in amounts as described above, but can also be added as solutions or added directly to the mixture. It is also believed that lower amounts of the activated salts can be used to achieve the desired effects that have usually required higher amounts of regular salts or activated salts having larger particle sizes.

With respect to various types of formulations in which the activated salts of this invention may be useful, the following types are included. These formulations may be viewed as suspensions or emulsions. The physical forms of these formulations include sticks, gels, creams, soft solids, roll-ons, pump sprays and aerosols. Representative formulations include the following:
(a) silicone based soft solid formulae where the systems are thickened with waxes, silicas, elastomers, clays and other thickening agents;
(b) anhydrous sticks where the stick is gelled with fatty alcohols (for example, stearyl alcohol), polysiloxane polyamides, 12-hydroxy stearic acids, waxes or binders,
(c) pump sprays where the active is suspended in a suitable vehicle; and
(d) aerosols where the active is suspended in a suitable vehicle (such as cyclomethicone) and a hydrocarbon or hydrofluorocarbon propellant (such as blended butanes) is used.

More specific formulations include:

### Stick:

0.5-25% enhanced active salt made by the method of this invention, 20-80% cyclomethicone, 5-80% wax (for example castor wax, stearyl alcohol or beeswax); 0-20% surfactant (for example, ethoxylated and/or propoxylated materials such as PPG-14 butyl ether);
0-50% emollients (for example fatty esters having 6-18 carbons, hydrocarbons such as petrolatum,); and 0-3% fragrance.

### Soft Solid:

0.5-25% enhanced active salt made by the method of this invention; 20-80% cyclomethicone; 5-80% wax (for example castor wax, stearyl alcohol or beeswax); 0-20% surfactant (for example, ethoxylated and/or propoxylated materials such as PPG-14 butyl ether); 0-50% emollients (for example fatty esters having 6-18 carbons, hydrocarbons such as petrolatum,); 0-3% fragrance; 0-10% clay (for example laponite or bentonites); 0-60% inert filled (for example, polyethylene, polypropylene, polytetrafluoroethylene, starch and/or talc).

### Roll-on:

20-90% cyclomethicone; 0-20% dimethicone (up to 350 centistokes); 0-10% quaternium-18 hectorite; 0.5-25% enhanced active made by the method of this invention; and 0-3% fragrance.

### Aerosol:

5-30% cyclomethicone; 0-20% dimethicone (up to 350 centistokes); 0-10% quaternium-18 hectorite; 0.5-25% enhanced active made by the method of this invention; 50-80% propellant (for example, blended butanes); and 0-3% fragrance.
Pump Spray: Aerosol formulation without the propellant.

The formulations made according to this invention are normally opaque.

The formulations of this invention may be made with out the use of a surfactant.

An important feature of this invention is the ability to obtain products with improved efficacy and aesthetics. This may be viewed as improvement in four aspects:

(a) the increase of the amount of smaller species of aluminum and zirconium which is known to increase efficacy;

(b) the ability to obtain better coverage of the underarm area with the same amount of salt (better and more even distribution);

(c) the improvement of the active's affinity for skin; and

(d) better aesthetics.

More particularly, the release of antiperspirant actives into the sweat is a significant event in the development of an antiperspirant effect. The magnitude of the antiperspirant effect is related to the concentration of the antiperspirant salt in the sweat concentration. It is well known that the smaller species are more desirable that the larger species in terms of antiperspirant activity. (See Antiperspirants and Deodorants, edited by Karl Laden, second edition, (Marcel Dekker, Inc., N.Y., N.Y. 1999), especially Chapter 4.)

The ability of the enhanced salt to act as an antiperspirant active was verified by diluting a solution of an enhanced active as made by the method of the invention in water and observing the reformation of the Peaks assigned to the larger Al and Zr species (Peak 1 for zirconium and Peak 3 for aluminum).

The cosmetic composition according to the present invention can be packaged in conventional containers, using conventional techniques. For example, where the composition is a stick composition, the composition, while still in liquid form, can be introduced into a dispensing package as conventionally done in the art, and cooled therein so as to thicken in the package. Where a gel or soft-solid cosmetic composition is produced, the composition can be introduced into a dispensing package (for example, a package having a top surface with pores) as conventionally done in the art. Thereafter, the product can be dispensed from the dispensing package as conventionally done in the art, to deposit the active material, for example, on the skin. This provides good deposition of the active material on the skin.

Throughout the present specification, where compositions are described as including or comprising specific components or materials, or where methods are described as including or comprising specific steps, it is contemplated by the inventors that the compositions of the present invention also consist essentially of, or consist of, the recited components or materials, and also consist essentially of, or consist of, the recited steps. Accordingly, throughout the present disclosure any described composition of the present invention can consist essentially of, or consist of, the recited components or materials, and any described method of the present invention can consist essentially of, or consist of, the recited steps.

As mentioned previously, the present invention includes within its scope (but is not limited to) creams, “soft gels” and sticks. The stick form can be distinguished from a soft gel in that, in a stick, the formulated product can maintain its shape for extended time periods outside the package, the product not losing its shape significantly (allowing for some shrinkage due to solvent evaporation). Soft gels can be suitably packaged in containers which have the appearance of a stick, but which dispense through apertures (for example, slots or pores) on the top surface of the package.

In the cosmetics field, systems are classified as soft gels or sticks, depending on their viscosity or hardness alone; typically, it is understood that soft gels are soft, deformable products while sticks are strictly free-standing solids. For example, by rheological analysis, a commercial deodorant stick has been determined to have a plateau storage modulus G′(ω) of roughly 10⁵ Pa and a complex viscosity of 10⁶ Pa second, both at an angular frequency of 0.1 rad/sec). On the other hand, a commercial antiperspirant soft gel has been determined to have a G′(ω) value of roughly 10³ Pa and a complex viscosity of 10⁴ Pa second (at 0.1 rad/sec). Use of the present glycol component provides particularly good results in connection with soap-based compositions (for example, deodorant gel compositions gelled utilizing a soap gelling agent).

The following Examples are offered as illustrative of the invention and are not to be construed as limitations thereon. In the Examples and elsewhere in the description of the invention, chemical symbols and terminology have their usual and customary meanings. Temperatures are in degrees Celsius unless otherwise indicated. The amounts of the components in the Examples as well as elsewhere in the application, are in weight percents based on the standard described; if no other standard is described then the total weight of the compositions is to be inferred. Various names of chemical components used in this application include those listed in the CTFA International Cosmetic Ingredient Dictionary (Cosmetics Toiletry and Fragrance Association, Inc. 4^{th} ed. 1991).

### EXAMPLES

### Process Examples

### Example 1P

### General Process

One method of how an antiperspirant salt (ACH or ZAG) is ground in order to enhance small aluminum and zirconium polymeric species is as follows. The premix is made up with 25% solid (w/w) by adding 500 gm of the anhydrous salt powder into 1500 gm of cyclomethicone (D5), and stirring the slurry to make a uniform suspension. The salt suspension is processed on the LabStar I Zeta mill (NETZSCH Inc., Exton, Pa.). The Zeta mill has silicon carbide wetted parts (shaft and chamber) with a screen size of 0.2 mm, and is loaded with a 90% charge of 0.4 mm YTZ (Yttrium coated ZrO₂ beads) as grinding media about 1.5 kg). The salt suspension is re-circulated at an average rate of 0.75 kg/min, and the agitator speed is maintained around 3000 RPM. The temperature of the suspension is controlled to stay below 60° C. by passing chilled water (4° C.) at a flow rate of 1/min in a jacket around the vessel. The particle size distribution of the dispersed salt powder is measured with LA-900 Laser Scattering Particle Size Distribution Analyzer (Horiba Instruments, Inc. Irvine, Calif.) every 30 minutes. The ground sample is also collected to analyze the molecular weight distribution of the metal polymers by SEC (Size Exclusion Chromatography) as described in Example 1S.

### Example 2P

The process of Example 1P may be repeated with the following changes. The shaft is polyurethane, the bead size used in 0.2 mm, the screen size used is 0.1 mm with more open surface area, and the agitator speed is about 3200 RPM.

### Salt Examples

### Example 1S

### General Analytical Technique

SEC (Size Exclusion Chromatography) analysis is the primary technique used in this invention for characterizing ZAG salts in terms of separating, detecting and measuring zirconium and aluminum polymer species. The chromatogram is run using the following parameters: Waters® 600 analytical pump and controller, Rheodvne® 77251 injector, Protein-Pak® 125 (Waters) column, Waters 996 Photodiode Array Detector at a wavelength of 240 nm, 5.56 mM nitric acid mobile phase, 0.70 ml/min flow rate, 2.0 microliter injection volume. Data was analyzed using Waters® millenium 2.1 software (Waters Corporation, Milford, Mass.). At least five distinguished peaks can be shown for a ZAG sample, each identified by a distribution coefficient (Kd) as follows: Peak 1 (Kd=0), Peak 2 (Kd=0.05), Peak 3 (Kd=0.20), Peak 4 (Kd=0.33) and Peak 5 (or Peak 5 & 6) (Kd=0.53), which is defined by the equation:

Kd=(Ve−Vo)/(Vt−Vo)

where: Ve=elution volume of peak
Vo=exclusion volume of column
Vt=total volume of column

For SEC analysis of a sample of ground salt suspension as made by the method described in Example 1P, the non-aqueous liquid vehicle is removed by means of centrifugation (3900 RPM), the salt is then dissolved in distilled water to make a 10% (w/w) solution, and the solution is used for injection onto the column.

The increase in smaller aluminum species is calculated by obtaining the values for

$$\frac{Peak 4 area + Peak 5 area}{Peak 3 area + Peak 4 area + Peak 5 area} before grinding = {P}_{before}$$

$$\frac{Peak 4 {area}^{′} + Peak 5 {area}^{′}}{\begin{matrix}Peak 3 {area}^{′} + Peak 4 {area}^{′} + \\ Peak 5 {area}^{′}\end{matrix}} after grinding = {P}_{after} \\ ( {P}_{after} - {P}_{before} ) \times 100 = \% increase$$

where the values marked “′” are those taken after grinding.

The decrease in larger zirconium species is obtained by calculating the decrease in the area of Peak 1 as

$$\frac{\begin{matrix}Peak 1 area before grinding - \\ Peak 1 area after grinding\end{matrix}}{Peak 1 area before grinding} \times 100 = \% decrease$$

The method described in Example 1P or 2P may be used to obtain the following salts with the method of Example 1S being used to evaluate the increase in the smaller aluminum species the decrease in the larger zirconium species.

### Example 2S

The method of Example 1P was used to obtain an enhanced salt as evaluated by the method of Example 1S. A sample of Reach AZP-908 (from Reheis Inc. 235 Snyder Ave., Berkeley Heights, N.J. 07922) 25% in cyclomethicone was ground for 90 minutes using the method described in Example 1P with the following results (μ=microns).

TABLE 1
Particle size distribution of AZP-908 powder suspended in
cyclomethicone

| | | | 99% of the particles |
|---|---|---|---|
| Status | mean | median | smaller than |
| Before grinding | 5.882μ | 5.426μ | 14.856μ |
| After 30 min. grinding | 1.941μ | 1.815μ | 14.856μ |
| After 60 min. grinding | 1.452μ | 1.395μ | 4.202μ |
| After 90 min. grinding | 1.114μ | 1.100μ | 2.131μ |

See FIG. 1 as SEC chromatograms for the ground AZP-908.

TABLE 2
SEC analysis for ground AZP-908 (Peak area distribution)

| Status | Peak 1 | Peak 3 | Peak 4 | Peak 5 |
|---|---|---|---|---|
| Before grinding | 35.5% | 39.9% | 7.5% | 17.1% |
| After grinding 30 min. | 24.1% | 41.0% | 11.8% | 23.1% |
| (Mean: 1.941μ) | | | | |
| After grinding 60 min. | 17.3% | 40.3% | 14.3% | 28.2% |
| (Mean: 1.452μ) | | | | |
| After grinding 90 min. | 4.9% | 29.1% | 20.4% | 45.6% |
| (Mean: 1.142μ) | | | | |

TABLE 3
Peak area ratios indicating the change of polymer distribution

| Status | Peak 1/Peak 3 | Peak 4/Peak 3 | Peak 5/Peak 3 |
|---|---|---|---|
| Before grinding | 0.89 | 0.19 | 0.43 |
| After grinding 30 min. | 0.59 | 0.29 | 0.56 |
| (Mean: 1.941μ) | | | |
| After grinding 60 min. | 0.43 | 0.36 | 0.70 |
| (Mean: 1.452μ) | | | |
| After grinding 90 min. | 0.17 | 0.70 | 1.56 |
| (Mean: 1.142μ) | | | |

The increase in the amount of smaller aluminum species can be calculated as follows:

(1) proportion of smaller aluminum species in relation to all aluminum species in parent salt is: [(7.5+17.1)/(39.9+7.5+17.1)]×100=38%
(2) proportion of smaller aluminum species in relation to all aluminum species in salt after grinding is [(20.4+45.6)/(29.1+20.4+45.6)]×100=69%
(3) increase in amount of smaller aluminum species is 69%-38%=31%

The increase in the amount of smaller zirconium species can be calculated as follows:

Area for Peak 1 before grinding=35.5−Area for Peak 1 after grinding=4.9. (1)

35.5−4−9=30.6 (2)

[30.6/35.5]×100=86% reduction in large zirconium species. (3)

### Example 3S

The method of Example 1P was used to obtain an enhanced salt as evaluated by the method of Example 1S. A sample of Reach AZZ-902 (from Reheis Inc.) 25% in cyclomethicone was ground for 90 minutes using the method described in Example 1P with the following results.

TABLE 4
Particle size distribution of AZZ-902 powder suspended in
cyclomethicone

| | | | 99% of the particles |
|---|---|---|---|
| Status | mean | median | smaller than |
| Before grinding | 5.647μ | 5.182μ | 12.982μ |
| After 90 min. grinding | 1.036μ | 1.014μ | 1.709μ |

See FIG. 2 for SEC chromatograms for the ground Reach AZZ-902.

TABLE 5
SEC analysis for ground Reach AZZ-902 (Peak area distribution)

| Status | Peak 1 | Peak 3 | Peak 4 | Peak 5 |
|---|---|---|---|---|
| Before grinding | 26.7% | 29.2% | 32.0% | 12.1% |
| After 90 min. grinding | 4.1% | 21.6% | 38.2% | 36.1% |

TABLE 6
Peak area ratios indicating the change of polymer distribution

| Status | Peak 1/Peak 3 | Peak 4/Peak 3 | Peak 5/Peak 3 |
|---|---|---|---|
| Before grinding | 0.91 | 1.09 | 0.41 |
| After 90 min. grinding | 0.19 | 1.80 | 1.68 |

In this Example 85% of the large Zr species were reduced and the amount of small A1 species was increased from 60% to 77%,

### Example 4S

The method of Example 1P was used to obtain an enhanced salt as evaluated by the method of Example 1S. A sample of Rezal-36 GP (from Reheis Inc.) 25% in cyclomethicone was ground for 60 minutes using the method described in Example 1P with the following results.

TABLE 7
Particle size distribution of Rezal-36 PG powder suspended in
cyclomethicone

| | | | 99% of the particles |
|---|---|---|---|
| Status | mean | median | smaller than |
| Before grinding | 6.731μ | 6.390μ | 15.005μ |
| After 90 min. grinding | 1.651μ | 1.576μ | 3.291μ |

See FIG. 3 for SEC chromatograms for the ground Rezal-36 PG.

TABLE 8
SEC analysis for ground Rezal-36 GP (Peak area distribution)

| Status | Peak 1 | Peak 3 | Peak 4 | Peak 5 |
|---|---|---|---|---|
| Before grinding | 35.4% | 32.3% | 8.7% | 23.6% |
| After 60 min. grinding | 16.7% | 35.1% | 15.5% | 32.7% |

TABLE 9
Peak area ratios indicating the change of polymer distribution

| Status | Peak 1/Peak 3 | Peak 4/Peak 3 | Peak 5/Peak 3 |
|---|---|---|---|
| Before grinding | 1.09 | 0.27 | 0.73 |
| After 60 min. grinding | 0.45 | 0.44 | 0.93 |

In this Example 53% of the large Zr species were reduced and the amount of small A1 species was increased from 50% to 58%. Note that in this Example dimethicone was used and the machine shut down after 45 minutes. A rerun of this example should include a viscosity modifier.

### Formulation Examples

The following formulations can be made with enhanced salts made according to his invention using the method and salts described above. A particular enhanced salt of interest is the one described in Example 2S which may be described as a ground active antiperspirant made with a 25% suspension of Reach AZP 902 in cyclcomethicone. The average particle size of this enhanced salt is 1.142 with at least 50% of the particles being 1.100 microns. All amounts are in percent by weight based on the entire weight of the composition. The enhanced salt is prepared by the wet grinding method of the invention.

### Example #1F

### Roll-On

A roll-on product may be made by combining the following ingredients with mixing until homogeneous:

| | |
|---|---|
| Enhanced salt (25% active/cyclomethicone) | 99% |
| Fragrance | 1% |

### Example #2F

### Soft Solid Without Elastomer

A soft solid product may be made by combining the following ingredients with mixing until homogeneous. Note that three formulations (3F, 4F, and 5F) are given.

| | |
|---|---|
| Enhanced Salt (25%) | 93.4% |
| Degussa R-812 Hydrophobically Modified Silica | 3.6% |
| Fragrance | 1.0% |

### Examples #3F-5F

### Soft Solid With Elastomer

Soft solid products may be made by combining the following ingredients with mixing until homogeneous. Note that three formulations (3F, 4F, and 5F) are given.

| Ingredient | 3F | 4F | 5F |
|---|---|---|---|
| Enhanced Salt (25%) | 66.0 | 50 | 33.0 |
| Shin Etsu KSG-15 Elastomer | 25 | 36.5 | 50.0 |
| (Shin Etsu Silicones of America, Akron, Ohio) | | | |
| AZZ902 Al-Zirconium trichlorohydrex | 8.0 | 12.5 | 16.0 |
| Fragrance | 1.0 | 1.0 | 1.0 |

### Example #6F

### No Residue Antiperspirant Stick

A soft solid product may be made by combining the following ingredients with mixing until homogeneous:

| | |
|---|---|
| Enhanced Salt (25%) | 58.7% |
| Stearyl Alcohol | 17.4% |
| PPG-14 Butyl Ether | 11.9% |
| Phenyl trimethicone | 5.0% |
| Hydrogenated Castor Oil | 4.0% |
| PEG-8 diisostearate | 2.0% |
| Fragrance | 1.0% |

Also, a mixed system may be used with regular salt and enhanced salt so that 52.2% of the enhanced salt (25% in cyclomethicone)+6.5% of an aluminum zirconium tetrachlorohydrex salt may be used.

### Example #7F

### Anhydrous Roll-On Antiperspirant

A roll-on product may be made by combining the following ingredients with mixing until homogeneous:

80.00% of a 25% suspension of an enhanced salt as described in any of the “S” Examples
9.00% C12-15 alkyl benzoate (Finsolv TN from Finetex, Inc., Elmwood Park, N.J.)
10.50% cyclomethicone (D5)
0.50% fragrance

### Example #8F

### Roll-On Antiperspirant (Suspension)

A roll-on suspension product may be made by combining the following ingredients with mixing until homogeneous:

24.00% cyclomethicone (D5)
1.40% of an aluminum zirconium trichlorohydrex gly antiperspirant salt
71.4% of a 25% suspension of an enhanced salt as described in any of the “S” Examples
3.00% quatemium-18 hectorite
1.00% propylene carbonate
0.50% fragrance
0.10% fumed silica

Also, a mixed system may be used with regular salt and enhanced salt so that 70.0% of the enhanced salt (25% in cyclomethicone)+1.40% of an aluminum zirconium trichlorohydrex salt may be used.

### Example #9F

### Antiperspirant Stick (No Residue)

A stick product may be made by combining the following ingredients with mixing, heating until all the waxes are solubilized, and until the whole mixture is homogeneous. The product is then poured into appropriate packages. 68.00% of a 25% suspension of an enhanced salt as described in any of the “S” Examples

14.00% stearyl alcohol
5.00% hydrogenated castor oil
0.50% fumed silica
0.50% fragrance
5.00% C12-15 alkyl benzoate
7.00% cyclomethicone (D5)

Also, a mixed system may be used with regular salt and enhanced salt so that 60.0% of the enhanced salt (25% in cyclomethicone)+8.00% of an aluminum zirconium trichlorohydrex salt may be used.

### Example #10F

### Antiperspirant Stick

A stick product may be made by combining the following ingredients with mixing, heating until all the waxes are solubilized, and until the whole mixture is homogeneous. The product is then poured into appropriate packages.

2.50% cyclomethicone (D5)
68.00% of a 25% suspension of an enhanced salt as described in any of the “S” Examples
3.00% PEG-8 distearate
8.00% hydrogenated castor oil
18.00% stearyl alcohol
0.50% fragrance

Also, a mixed system may be used with regular salt and enhanced salt so that 60.0% of the enhanced salt (25% in cyclomethicone)+8.00% of an aluminum zirconium trichlorohydrex salt may be used.

### Example #11F

### Wax Based Antiperspirant Cream

A cream product may be made by combining the following ingredients with mixing until homogeneous. No heating is required.

5.00% cyclomethicone (D5)
15.00% dimethicone (50 centistokes)
68.00% of a 25% suspension of an enhanced salt as described in any of the “S” Examples
6.50% hydrogenated castor oil
5.00% alkyl silicone wax (stearoxytrimethyl siloxane)
0.50% fragrance

Also, a mixed system may be used with regular salt and enhanced salt so that 60.0% of the enhanced salt (25% in cyclomethicone)+8.00% of an aluminum zirconium trichlorohydrex salt may be used.

### Example #12F

### Silicone Based Soft Solid Antiperspirant

A soft solid product may be made by combining the following ingredients with mixing until homogeneous:

70.00% of a 25% suspension of an enhanced salt as made by any of the “S” Examples described above
24.50% cyclomethicone and dimethicone crosspolymer (KSG-15 from Shin-Etsu)
5.00% C12-15 alkyl benzoate
0.50 fragrance

### Example #13F

### Wax Based Soft Solid Antiperspirant

A soft solid product may be made by combining the following ingredients with mixing until homogeneous:

60.00% of a 25% suspension of an enhanced salt as made by any of the “S” Examples described above
15.00% hexanediol behenyl beeswax
15.00% phenyl trimethicone
9.5% dimethicone (up to 350 centistokes, especially 200-350 cst)
0.50% fragrance

Also, a mixed system may be used with regular salt and enhanced salt so that 60.0% of the enhanced salt (25% in cyclomethicone)+10.00% of an aluminum zirconium trichlorohydrex salt may be used.

### Example #14F

### Wax Based Stick Antiperspirant

A stick product may be made by combining the following ingredients with mixing, heating until all the waxes are solubilized and until the whole mixture is homogeneous.

18.6% of an enhanced salt as made by any of the “S” Examples described above
55.8% cyclomethicone (D5)
22% stearyl alcohol
2% MP 90 castor wax
1% surfactant (PPG-14 butyl ether)
0.6% fragrance

### Example #15F

### Soft Solid Antiperspirant

A soft solid product may be made by combining the following ingredients with mixing until homogeneous:

25% of an enhanced salt as made by any of the “S” Examples described above
46% cyclomethicone (D5)
10.0% isocetyl alcohol
1% fragrance
5.0% quaternium-18 hectorite
13% starch (DRY FLO corn starch from National Starch, Finderne, N.J.)

### Example #16F

### Roll-On Antiperspirant

A roll-on product may be made by combining the following ingredients with mixing until homogeneous:

25% of an enhanced salt as made by any of the “S” Examples described above
66% cyclomethicone (D5)
5.0% dimethicone (200 centistokes)
3.0% quaternium-18 hectorite
1% fragrance

### Example #17F

### Aerosol Antiperspirant

An aerosol product may be made by combining the following ingredients with mixing until homogeneous:

20% of an enhanced salt as made by any of the “S” Examples described above
10% cyclomethicone (D5)
2% dimethicone (10 centistokes)
2% quaternium-18 hectorite
1% fragrance
65% propellant

We claim:

1. A method for enhancing the activity of an aluminum or an aluminum/zirconium salt containing small and large aluminum species, comprising
(a) combining a parent salt with a non-aqueous liquid vehicle in which the salt is suspended but not soluble to form a mixture; and
(b) grinding the mixture at a temperature in the range of 20-70° C. to an average particle size of less than or equal to 2 microns to form an enhanced salt;
wherein the enhancement comprises increasing the smaller species in an amount of at least 10% as compared to the parent salt and wherein the enhanced salt is characterized as having a sum of Peak 4+Peak 5 areas in the enhanced salt which is at least 10% greater than the sum of Peak 4+Peak 5 areas of the parent salt.

2. The method of claim 1, wherein the average particle size is less than or equal to 1.5 microns.

3. The method of claim 1, wherein the non-aqueous liquid is hydrophobic.

4. The method of claim 1, wherein the a non-aqueous liquid vehicle is selected from the group consisting of:
(a) fatty esters having 6-22 carbons in straight or branched chains;
(b) glycols and polyols;
(c) volatile silicones;
(d) non-volatile silicones having a viscosity of up to 350 centistokes;
(e) hydrocarbons;
(f) alcohols having more than three carbons;
(g) mixtures of the foregoing.

5. The method of claim 1, wherein the non-aqueous liquid vehicle is selected from the group consisting of:
(a) C12-C15 ethoxy benzoate
(b) Diethylene Glycol dioctanoate/diisononoate
(c) octyl palmitate
(d) diisopropyl adipate
(e) dipropylene glycol dibenzoate
(f) Glycereth-7 benzoate
(g) propylene glycol benzoate
(h) glycereth-7 polyurethane
(i) PPG-14 Butyl Ether
(j) PPG-26 oleate
(k) propylene glycol isoceteth-3 acetate
(l) Propylene Glycol Myristyl Ether Acetate
(m) D4-D6 cyclomethicones
(n) D5 cyclomethicone
(o) Methyl Gluceth-20 Benzoate
(p) Dipropylene glycol dibenzoate
(q) Poloxamer 105 Benzoate
(r) Poloxamer 182 Dibenzoate
(s) octyl stearate
(t) octocrylene
(u) PEG-4 diheptanoate
(v) neopentylglycol dicaprylate/dicaprate
(w) propylene glycol dicaprylate/dicaprate
(x) tridecyl stearate (y) glycereth-7 benzoate
(z) PPG-10 butane diol
(a-1) tetrabutoxy trisiloxane
(b-1) benzyl benzoate
(c-1) benzyl salicylate
(d-1) dipropylene glycol salicylate
(e-1) diisopropyl dimer dilinoleate
(f-1) diisostearyl dimer dilinoleate
(g-1) diisodecyl adipate
(h-1) Dioctyl sebacate
(i-1) tricapylin
(j-1) pentaerythritol tetraoctanoate
(k-1) dibutyl sebacate
(l-1) dicapryl adipate
(m-1) diisopropyl sebacate
(n-1) 2-ethylhexyl palmitate
(o-1) Dodecalene
(p-1) dipropylene glycol benzoate
(q-1) dioctyl succinate
(r-1) polydecene
(s-1) cetearyl octanoate
(t-1) isocetyl octanoate
(u-1) octyldodecyl myristate
(v-1) cetyl octanoate
(w-1) dicapryl malleate
(x-1) neopentyl dicaprate
(y-1) isostearyl neopentanoate
(z-1) Isocetyl stearoyl stearate
(a-2) Isostearyl stearoyl stearate
(b-2) neopentyl glycol dioctanoate/diisostearate
(c-2) neopentyl glycol diisostearate/dioctanoate
(d-2) PPG-15 Stearyl Ether Benzoate
(e-2) octyl dimethyl PABA
(f-2) octylmethoxycinnamate
(g-2) octyl dodecyl behenate
(h-2) cetyl octanoate
(i-2) isodecyl oleate
(j-2) 2-ethyl hexyl isostearate
(k-2) isostearyl isostearate
(l-2) isopropyl palmitate
(m-2) Isopropyl Stearate
(n-2) Octyl dodecyl myristate
(o-2) phenoxyethyl benzoate
(p-2) octyl isononanoate
(q-2) benzyl laurate/myristate/palmitate
(r-2) Isohexadecyl salicylate
(s-2) Isodecyl salicylate
(t-2) Isotridecyl salicylate
(u-2) octyl pelargonate
(v-2) triisocetyl citrate
(w-2) isodecyl tieopentanoate
(x-2) laureth-2 benzoate
(y-2) C12-C15 Alkyl Octanoate
(z-2) isoparaffin
(a-3) normal paraffin
(b-3) isododecane
(c-3) isoeicosatie
(d-3) isohexadecane
(e-3) Octyl Dodecyl Benzoate
(f-3) 2-ethyl hexyl benzoate
(g-3) isostearyl benzoate
(h-3) C₁₂-C₁₅ alkyl benzoate
(i-3) dicapryl ether
(j-3) hexyl laurate
(k-3) Dioctylcyclolhexane
(l-3) laureth-4
(m-3) C12-C145 alkyl lactate
(n-3) octyl salicylate
(o-3) C12/C14 alcohol mix
(p-3) isocetyl alcohol
(q-3) isostearyl alcohol
(r-3) C20 guerbet alcohol
(s-3) hexyl benzoate
(t-3) isononyl isononanoate
(u-3) isostearyl lactate
(v-3) benzyl laurate
(w-3) aliphatic hydrocarbons
(x-3) Tridecyl neopentanoate
(y-3) tridecyl octanoate
(z-3) octyl salycilate
(a-4) isopropyl isostearate
(b-4) isostearyl alcohol
(c-4) C12/C14 alcohol mix
(d-4) isopropyl isostearate
(e-4) light mineral oil
(f-4) petroleum distillate
(g-4) n-heptane
(h-4) 1-octanol
(i-4) lauryl alcohol
(j-4) isopropyl palmitate
(k-4) oleyl alcohol.

6. The method of claim 1, wherein the non-aqueous liquid vehicle is selected from the group consisting of cyclomethicones, mineral oils, and low viscosity fatty esters having 8-18 carbons.

7. The method of claim 6, wherein the non-aqueous liquid vehicle is cyclomethicone.

8. The method of claim 4, wherein the glycols and polyglycols are selected from the group consisting of ethylene glycol, propylene glycol, 1,2-propanediol, diethylene glycol, triethylene glycol, tetraethylene glycol, dipropylene glycol, tripropylene glycol, methyl propanediol, 1,6-hexanediol, 1,3-butanediol, 1,4-butanediol, PEG-4 through PEG-100, PPG-9 through PPG-34, pentylene glycol, neopentyl glycol, trimethylpropanediol, 1,4-cyclohexanedimethanol, 2,2-dimethyl-1,3-propane-diol, 2,2,4,4-tetramethyl-1,3-cyclobutanediol, and mixtures thereof.

9. The method of claim 4, wherein the glycols and polyglycols are selected from the group consisting of propylene glycol, dipropylene glycol, tripropylene glycol, 2-methyl-1,3-propanediol, methyl propylene glycol, low molecular weight (less than 600) polyethylene glycol, low molecular weight (less than 600) polypropylene glycols, and mixtures of any of the foregoing.

10. The method of claim 1, wherein the parent salt is selected from the group consisting of antiperspirant active salts containing aluminum and antiperspirant active salts containing both aluminum and zirconium.

11. The method of claim 10, wherein the parent salt is selected from the group consisting of zirconyl hydroxychliorides, zirconyl oxychlorides, basic aluminum chlorides, basic aluminum chlorides combined with zirconyl oxychlorides and hydroxychlorides, and organic complexes of each of basic aluminum chlorides with or without zirconyl oxychlorides and hydroxychlorides, and mixtures of any of the foregoing.

12. The method of claim 10, wherein the parent salt is selected from the group consisting of aluminum chlorohydrate, aluminum chloride, aluminum sesquichlorohydrate, aluminum chlorohydrol-propylene glycol complex, zirconyl hydroxychloride, aluminum-zirconium glycine complex, aluminum dichlorohydrate, aluminum chlorohydrex PG, aluminum chlorohydrex PEG, aluminum dichlorohydrex PG, aluminum dichlorohydrex PEG, aluminum zirconium trichlorohydrex gly propylene glycol complex, aluminum zirconium trichlorohydrex gly dipropylene glycol complex, aluminum zirconium tetrachlorohydrex gly propylene glycol complex, aluminum zirconium tetrachlorohydrex gly dipropylene glycol complex, and mixtures of any of the foregoing.

13. The method of claim 10, wherein the parent salt is selected from the group consisting of aluminum chlorohydrate, aluminum dichlorohyrate, aluminum sesquichlorohydrate, aluminum zirconium trichlorohyrate, aluminum zirconium tetrachlorohyrate, aluminum zirconium pentachlorohyrate, aluminum zirconium octachlorohyrate, aluminum zirconium trichlorohydrex gly, aluminum zirconium tetrachlorohydrex gly, and aluminum zirconium pentachlorohydrex gly.

14. The method of claim 10, wherein the parent salt is selected from the group consisting of aluminum chlorohydrate, aluminum chloride, aluminum sesquiclhlorohydrate, zirconyl hydroxychbloide, aluminum-zirconium glycine complex, aluminum chlorohydrex PG, aluminum chlorohydrex PEG, aluminum dichlorohydrex PG, and aluminum dichlorohydrex PEG.

15. The method of claim 10 wherein the parent salt is selected from the group consisting of aluminum zirconium trichlorohydrex and aluminum zirconium tetrachlorohydrex either with or without glycine.

16. An enhanced salt as obtained by the method of claim 1 wherein the enhanced salt is characterized as having a sum of Peak 4+Peak 5 areas in the enhanced salt which is at least 20% greater than the sum of Peak 4+Peak 5 areas of the parent salt.

17. An enhanced salt as obtained by the method of claim 1 wherein the parent salt comprises aluminum and zirconium and the enhanced salt is further characterized as having a Peak 1 area of at least 10% less than the Peak 1 area of the parent salt.
